SEQUENCE LISTING (4 sequences)

SEQ ID NO: 1 - DNA, 4493 bp, Homo sapiens
  CDS (380)..(2179)
agcgggaggggttgcgggtgagctgcgctgagcccaggagccgaggagtcgggagcgcagcgcagcccgagcccgagcccgagcggccccgcgtcccgagcgcatcggagcggccgagccgcccggcgcagcgcctggcccgggcagcgcagccccggccgcaggatctcaccaaaggtggcagaaggaggccttctggagctgacccacccccgacgaccatcagggtgaggcaactccaaggtcctactctctttctgtgcctgttacccaccccgtcctcctagggtgcccttgagccgcaaaactgctgtccacgtggaccgggggtgacatcgcacgtccatctgccaggacccctgcgtccaaattccgagacatggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcacgaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgcaactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagggtgggggccactcaccgacccgacactctcaccccccgacctggctgagtgcgaccaccacttgatgtctgaggataccttccatctcaacctacctcgagtggcgagtccagacaccatcaccacgcagagaggggaggtgggaggacagttagacccctgggtgggccctgccgtgggcaaggatacccggtggcttctggcacctggcgggctggtgacctttttaatccaggccccatcagcatcccacgatcggccttggtaaccgccgcggtagatcatttttatcccgccagggagtgtgatgcaggaagaccacatgcgctcctggcttttaaacctgttcctgactgttctcttactgccgaaacccttgactgttatctcggactttgcaggagttcctttccctccgaacgctgctccatgcacaggaaaagggcattttgtacaatggggacttcccgggaacgcttgctcttaagtaccagaagccggcggagctctggctttcgtgtttttggttttctccttcccaaggcagctggattgaaaaaacaaaacaaaacaaaaaaacccaggggcgtcagtcgatattcccagggccgcttctcctgcagtctgtggagcgtccttgtccccgccgccggaatgaatgagcattctgcagcccgatgtccctgtcccctcctcgccgggccattctgattggacctggcccagtgcaatctgtccagacaagccctgcttgctggaaaactgccacaagcacaattgatctctttttatcgccattccaggggcctcaggtcctactggggaaacttcctataccggagctccagtttctcttaagctgcccaatttcacagagtacaaaatagttgtaggggaaatcaaggtgaaggatctgtccgacagtcaagacggatccacagtaatctttcggtctccttaaactaccaccctcgctgccacccaccccaagctgctgccgcctcaccttccttgaaatttctcagcgggagtctcctcactgccactaaaacccacccagcccactaactgaggagctagtgttaatccagagaaccccccgcaatgtgcttccgagattcagactgcttcattgggaagtatgatttgttcctttctggaattgggctccgtggtggcggcggcacttcaagcaaagacagtttcttgcaagctccagtagctccgcgtgtctcatttgccaggaagatgggttcccacgtagcaaatcgtacattgtgccctgtagctccttagctagttagctcacaagccgtgttttatgactaatccttaataactatggtaaataactgtgactgtggggtttttaatctcttgtcattctcatccaaaagtgaccagcataccagttcttgcaataagatattaccctcagaatattaagcacattattgtagagaaaaaaaaatatgtgtacacatatgaacgcacaacatgcacattcatcatcacatgtggcacgtaaggtctcatttgatattgtgtaggaaatctgaagccttttcctgaggtcatctgtaaaatagtctcattgccaaggcatccccagtgccagctggtgaatccatgatcaaaatgcatacgtattgttaaatgataaggtttagaatgacaggaacccatcactgtgtctcatggtcccacttccccatctgtgtgtgaattcctttagactaagggcaggaagacttccagctttctctttgttcttcaatgtgaaactgagaccaagtctctctaagacaaatgcagtgtatttaatgtttgtaagcaattctaagtgagatgtttggcaagaaatcccctaactgatttccatccaaacctaccttatagagcacaatattaagtgttgtacaattactgtgagaactgtgaatatgtgtaacttttttttagtatttgcccggggggaaaaagatattgtattatcatatatgctttttttgcaataaggatttattctcagaacaccaagtaaatctatctctatataaaaaatatatgtaatatatacatattcaaagtatatacagagcctgttttaaaaaatacagtattatttagtaaaattatctgttctatggaccaaatgtaaaatatttataaatgaagatgcattttaaatgtctataaatggtgtcataactagagcacgggcgttatgtaagtttctaagaatttagaggataaataataaaggttctatgatatacaacaaaaaaaaaaaaaagaaaaaaaaaaaaaaaa

SEQ ID NO: 2 - protein, 599 aa, Homo sapiens
MATNGSKVADGQISTEVSEAPVANDKPKTLVVKVQKKAADLPDRDTWKGRFDFLMSCVGYAIGLGNVWRFPYLCGKNGGGAFLIPYFLTLIFAGVPLFLLECSLGQYTSIGGLGVWKLAPMFKGVGLAAAVLSFWLNIYYIVIISWAIYYLYNSFTTTLPWKQCDNPWNTDRCFSNYSMVNTTNMTSAVVEFWERNMHQMTDGLDKPGQIRWPLAITLAIAWILVYFCIWKGVGWTGKVVYFSATYPYIMLIILFFRGVTLPGAKEGILFYITPNFRKLSDSEVWLDAATQIFFSYGLGLGSLIALGSYNSFHNNVYRDSIIVCCINSCTSMFAGFVIFSIVGFMAHVTKRSIADVAASGPGLAFLAYPEAVTQLPISPLWAILFFSMLLMLGIDSQFCTVEGFITALVDEYPRLLRNRRELFIAAVCIISYLIGLSNITQGGIYVFKLFDYYSASGMSLLFLVFFECVSISWFYGVNRFYDNIQEMVGSRPCIWWKLCWSFFTPIIVAGVFIFSAVQMTQLTMGNYVFPKWGQGVGWLMALSSMVLIPGYMAYMFLTLKGSLKQRIQVMVQPSEDIVRPENGPEQPQAGSSTSKEAYI

SEQ ID NO: 3 - DNA, 3982 bp, Homo sapiens
  CDS (35)..(1933)
agccgggccggcgcacgaggcagccagcgcggccatgacggcggagaaggcgctgcccctgggcaatgggaaggctgctgaggaggcgcgggagtccgaggcgccgggtggcggctgcagcagcgggggcgcggcgcccgcgcgccacccgcgcgtcaagcgcgacaaggcggtccacgagcgcggccactggaacaacaaggtggagttcgtgctgagcgtggccggggagatcattgggctgggcaacgtgtggcgcttcccctacctgtgctacaagaacggaggaggggcattcctgattccctacgtggtgttttttatttgctgtggaattcctgtttttttcctggagacagctctggggcagttcacaagtgaaggtggcattacgtgttggaggaaagtttgccctttatttgaaggcattggctatgcaacacaggtgattgaggcccatctgaatgtgtactacatcatcatcctggcatgggccattttttacctgagcaactgcttcactactgagctaccctgggctacctgtgggcatgagtggaacacagagaattgtgtggagttccagaaactgaatgtgagcaactacagccatgtgtctctgcagaatgccacctcccctgtcatggagttttgggagcaccgggtcctggccatctctgacgggatcgagcacatcgggaaccttcgctgggagctggccttgtgtctcttggcagcctggaccatctgttacttctgtatctggaaggggaccaagtctacaggaaaggttgtatacgtgactgcgacattcccctacatcatgctgctgatcctcctgatacgaggggtcacgttgcccggggcctcagagggcatcaagttctacttgtaccctgacctctcccggctctccgacccccaggtctgggtagatgctggaacgcagatctttttctcctatgccatttgcctgggctgtctgaccgctctgggaagttataacaattataacaacaactgctacagggactgcatcatgctctgttgcctgaacagcggcaccagcttcgtggctgggtttgccatcttctcagtcctgggttttatggcgtacgagcagggggtacccattgctgaggtggcagagtcaggccccggcctggcctttattgcgtaccccaaggcggtcaccatgatgcctctctccccgctgtgggccaccttgttcttcatgatgctcatcttcctgggcctggacagccagtttgtgtgtgtggaaagcctggtgaccgccgtggtggacatgtaccccaaggttttccggaggggttaccggcgggagctgctcatcctagccttgtctgttatctcctattttctgggcctcgtgatgttaacagagggtggcatgtacatcttccagctctttgactcctatgccgccagtgggatgtgccttctcttcgtggccatctttgagtgcatctgcatcggctgggtgtatggaagcaaccggttctatgataacattgaagacatgattggctaccggccaccgtcgctcattaagtggtgctggatgatcatgacccctgggatctgcgcggggatcttcatcttcttcttgatcaagtacaagccactcaagtacaacaacatctacacctacccagcctggggctatggcattggctggctcatggccctgtcctccatgctctgcatcccgctctggatctgcatcacagtgtggaagacggaggggacactgcccgagaaactccagaagttgacgacccccagcacagatctgaaaatgcggggcaagcttggggtgagcccacggatggtgacagttaatgactgtgatgccaaactcaagagtgacgggaccatcgcagccatcacagagaaggagacgcacttctgagcggccaccagccatctggggctcttcttcctttcttccccccgtgtatgtaaatgaaagccgggccggcgcacgaggcagccagcgcggccatgacggcggagaaggcgctgcccctgggcaatgggaaggctgctgaggaggcgcgggagtccgaggcgccgggtggcggctgcagcagcgggggcgcggcgcccgcgcgccacccgcgcgtcaagcgcgacaaggcggtccacgagcgcggccactggaacaacaaggtggagttcgtgctgagcgtggccggggagatcattgggctgggcaacgtgtggcgcttcccctacctgtgctacaagaacggaggaggggcattcctgattccctacgtggtgttttttatttgctgtggaattcctgtttttttcctggagacagctctggggcagttcacaagtgaaggtggcattacgtgttggaggaaagtttgccctttatttgaaggcattggctatgcaacacaggtgattgaggcccatctgaatgtgtactacatcatcatcctggcatgggccattttttacctgagcaactgcttcactactgagctaccctgggctacctgtgggcatgagtggaacacagagaattgtgtggagttccagaaactgaatgtgagcaactacagccatgtgtctctgcagaatgccacctcccctgtcatggagttttgggagcaccgggtcctggccatctctgacgggatcgagcacatcgggaaccttcgctgggagctggccttgtgtctcttggcagcctggaccatctgttacttctgtatctggaaggggaccaagtctacaggaaaggttgtatacgtgactgcgacattcccctacatcatgctgctgatcctcctgatacgaggggtcacgttgcccggggcctcagagggcatcaagttctacttgtaccctgacctctcccggctctccgacccccaggtctgggtagatgctggaacgcagatctttttctcctatgccatttgcctgggctgtctgaccgctctgggaagttataacaattataacaacaactgctacagggactgcatcatgctctgttgcctgaacagcggcaccagcttcgtggctgggtttgccatcttctcagtcctgggttttatggcgtacgagcagggggtacccattgctgaggtggcagagtcaggccccggcctggcctttattgcgtaccccaaggcggtcaccatgatgcctctctccccgctgtgggccaccttgttcttcatgatgctcatcttcctgggcctggacagccagtttgtgtgtgtggaaagcctggtgaccgccgtggtggacatgtaccccaaggttttccggaggggttaccggcgggagctgctcatcctagccttgtctgttatctcctattttctgggcctcgtgatgttaacagagggtggcatgtacatcttccagctctttgactcctatgccgccagtgggatgtgccttctcttcgtggccatctttgagtgcatctgcatcggctgggtgtatggaagcaaccggttctatgataacattgaagacatgattggctaccggccaccgtcgctcattaagtggtgctggatgatcatgacccctgggatctgcgcggggatcttcatcttcttcttgatcaagtacaagccactcaagtacaacaacatctacacctacccagcctggggctatggcattggctggctcatggccctgtcctccatgctctgcatcccgctctggatctgcatcacagtgtggaagacggaggggacactgcccgagaaactccagaagttgacgacccccagcacagatctgaaaatgcggggcaagcttggggtgagcccacggatggtgacagttaatgactgtgatgccaaactcaagagtgacgggaccatcgcagccatcacagagaaggagacgcacttctgagcggccaccagccatctggggctcttcttcctttcttccccccgtgtatgtaaatgaa

SEQ ID NO: 4 - protein, 632 aa, Homo sapiens
MTAEKALPLGNGKAAEEARESEAPGGGCSSGGAAPARHPRVKRDKAVHERGHWNNKVEFVLSVAGEIIGLGNVWRFPYLCYKNGGGAFLIPYVVFFICCGIPVFFLETALGQFTSEGGITCWRKVCPLFEGIGYATQVIEAHLNVYYIIILAWAIFYLSNCFTTELPWATCGHEWNTENCVEFQKLNVSNYSHVSLQNATSPVMEFWEHRVLAISDGIEHIGNLRWELALCLLAAWTICYFCIWKGTKSTGKVVYVTATFPYIMLLILLIRGVTLPGASEGIKFYLYPDLSRLSDPQVWVDAGTQIFFSYAICLGCLTALGSYNNYNNNCYRDCIMLCCLNSGTSFVAGFAIFSVLGFMAYEQGVPIAEVAESGPGLAFIAYPKAVTMMPLSPLWATLFFMMLIFLGLDSQFVCVESLVTAVVDMYPKVFRRGYRRELLILALSVISYFLGLVMLTEGGMYIFQLFDSYAASGMCLLFVAIFECICIGWVYGSNRFYDNIEDMIGYRPPSLIKWCWMIMTPGICAGIFIFFLIKYKPLKYNNIYTYPAWGYGIGWLMALSSMLCIPLWICITVWKTEGTLPEKLQKLTTPSTDLKMRGKLGVSPRMVTVNDCDAKLKSDGTIAAITEKETHF

(19) United States
(12) Patent Application Publication
(10) Pub. No.: US 2008/0213765 A1
(43) Pub. Date: Sep. 4, 2008

(54) Human Autism Susceptibility Genes Encoding a Neurotransmitter Transporter and Uses Thereof

(75) Inventors: Anne Philippi, St Fargeau Ponthierry (FR); Francis Rousseau, Savigny Sur Orge (FR); Elke Roschmann, Beimerstetten (DE)

Correspondence Address: OCCHIUTI ROHLICEK & TSAO, LLP, 10 FAWCETT STREET, CAMBRIDGE, MA, 02138, US

(73) Assignee: Integragen, Genavenir (FR)

(21) Appl. No.: 11/817,291

(22) PCT Filed: Feb. 27, 2006
(86) PCT No.: PCT/IB2006/001100

§ 371 Date: Aug. 28, 2007

Related U.S. Application Data
(60) Provisional application No. 60/656,374, filed on Feb. 28, 2005.

(51) Int. Cl. C12Q 1/68; G01N 33/53

(52) U.S. Cl. 435/6; 435/7.1

(57) ABSTRACT

The present invention discloses the identification of a human autism susceptibility gene, which can be used for the diagnosis, prevention and treatment of autism and related disorders, as well as for the screening of therapeutically active drugs. The invention more specifically discloses that the SLC6A1 or SLC6A11 gene on chromosome 3 and certain alleles thereof are related to susceptibility to autism and represent novel targets for therapeutic intervention. The present invention relates to particular mutations in the SLC6A1 or SLC6A11 gene and expression products, as well as to diagnostic tools and kits based on these mutations. The invention can be used in the diagnosis of predisposition to, detection, prevention and/or treatment of Asperger syndrome, pervasive developmental disorder, childhood disintegrative disorder, mental retardation, anxiety, depression, attention deficit hyperactivity disorders, speech delay, epilepsy, metabolic disorder, immune disorder, bipolar disease and other psychiatric and neurological diseases including schizophrenia.

### FIELD OF THE INVENTION

The present invention relates generally to the fields of genetics and medicine.

### BACKGROUND OF THE INVENTION

Autism is a neuropsychiatric developmental disorder characterized by impairments in reciprocal social interaction and verbal and non-verbal communication, restricted and stereotyped patterns of interests and activities, and the presence of developmental abnormalities by 3 years of age (Bailey et al., 1996). In his pioneer description of infantile autism, Kanner (1943) included the following symptoms: impaired language, lack of eye contact, lack of social interaction, repetitive behavior, and a rigid need for routine. He noted that in most cases the child's behavior was abnormal from early infancy. On this basis, he suggested the presence of an inborn, presumably genetic, defect. One year later, Hans Asperger in Germany described similar patients and termed the condition “autistic psychopathy”.

Autism is defined using behavioral criteria because, so far, no specific biological markers are known for diagnosing the disease. The clinical picture of autism varies in severity and is modified by many factors, including education, ability and temperament. Furthermore, the clinical picture changes over the course of the development within an individual. In addition, autism is frequently associated with other disorders such as attention deficit disorder, motor in coordination and psychiatric symptoms such as anxiety and depression. There is some evidence that autism may also encompass epileptic, metabolic and immune disorder. In line with the clinical recognition of the variability, there is now general agreement that there is a spectrum of autistic disorders, which includes individuals at all levels of intelligence and language ability and spanning all degrees of severity.

Part of the autism spectrum, but considered a special subgroup, is Asperger syndrome (AS). AS is distinguished from autistic disorder by the lack of a clinically significant delay in language development in the presence of the impaired social interaction and restricted repetitive behaviors, interests, and activities that characterize the autism spectrum disorders (ASDs).

ASDs are types of pervasive developmental disorders (PPD). PPD, “not otherwise specified” (PPD-NOS) is used to categorize children who do not meet the strict criteria for autism but who come close, either by manifesting atypical autism or by nearly meeting the diagnostic criteria in two or three of the key areas.

To standardize the diagnosis of autism, diagnostic criteria have been defined by the World Health Organisation (International Classification of Diseases, 10^{th} Revision (ICD-10), 1992) and the American Psychiatric Association (Diagnostic and Statistical Manual of Mental Disorders, 4th edition (DSM-IV), 1994). An Autism Diagnostic Interview (ADI) has been developed (Le Couteur et al., 1989; Lord et al., 1994). The ADI is the only diagnostic tool available to diagnose ASD that has been standardized, rigorously tested and is universally recognized. The ADI is a scored, semi-structured interview of parents that is based on ICD-10 and DSM-IV criteria for the diagnosis of autism. It focuses on behavior in three main areas: qualities of reciprocal social interaction; communication and language; and restricted and repetitive, stereotyped interests and behaviors. Using these criteria, autism is no longer considered a rare disorder. Higher rates of 10-12 cases per 10,000 individuals have been reported in more recent studies (Gillberg and Wing, 1999) compared to the previously reported prevalence rate of 4-5 patients per 10,000 individuals based on Kanner's criteria (Folstein and Rosen-Sheidley, 2001). Estimates for the prevalence rate of the full spectrum of autistic disorders are 1.5 to 2.5 times higher. Reports of a four times higher occurrence in males compared to females are consistent. Mental retardation is present in between 25% and 40% of cases with ASD (Baird et al. 2000; Chakrabarti and Fombonne, 2001). Additional medical conditions involving the brain are seen in ca. 10% of the population (Gillberg and Coleman, 2000).

The mechanisms underlying the increase in reported cases of autism are unknown. It is highly debated whether this difference reflects an increase in the prevalence of autism, a gradual change in diagnostic criteria, a recognition of greater variability of disease expression, or an increased awareness of the disorder. In addition, there is a widespread public perception that the apparent increase is due primarily to environmentally factors (Nelson, 1991; Rodier and Hyman, 1998). However, it seems likely that most of the increased prevalence can be explained by a broadening of the diagnostic criteria, in combination with a broader application of these criteria.

Although there are effective treatments for ameliorating the disease, there are no cures available and benefits of treatment tend to be modest. Promising results have been obtained for several programs utilizing various behavioral and developmental strategies. Among the most promising are programs based on applied behavior analysis (ABA). Several medications appeared to improve various symptoms associated with autism, thereby increasing individuals' ability to benefit from educational and behavioral interventions. The most extensively studied agents are the dopamine antagonists. Several studies suggest the usefulness of various selective serotonin reuptake inhibitors.

Three twin studies have been performed to estimate heritability of autism (Folstein and Rutter, 1977; Bailey et al., 1995; Steffenburg et al., 1989). All twins who lived in a geographically defined population were sought out. In the combined data 36 monozygotic (MZ) and 30 dizygotic (DZ) twins were studied. The average MZ concordance rate is 70% compared to a DZ rate of 0%. A heritability of more than 90% was calculated from the MZ to DZ concordance ratio and the sibling recurrence risk that has been estimated to be ca 2%-4% (Jorde et al., 1991 Szatmari et al., 1998). Studies of non-autistic relatives have clearly shown that several characteristics of the ASDs are found more often in the parents of autistic children than the parents of controls including social reticence, communication difficulties, preference for routines and difficulty with change (Folstein and Rutter, 1977). Delayed onset of speech and difficulty with reading are also more common in family members of individuals with autism, as are recurrent depression, anxiety disorders, elevated platelet serotonin and increased head circumference (Folstein and Rosen-Sheidley, 2001).

The incidence of autism falls significantly with decreasing degree of relatedness to an affected individual indicating that a single-gene model is unlikely to account for most cases of autism (Jorde et al., 1990). A reported segregation analysis was most consistent with a polygenic mode of inheritance (Jorde et al., 1991). The most parsimonious genetic model is one in which several genes interact with one another to produce the autism phenotype (Folstein and Rosen-Sheidley, 2001).

Considerable indirect evidence indicates a possible role for autoimmunity in autism. One study found more family members with autoimmune diseases in families with an autistic proband compared with control probands (Comi et al., 1999). A few studies reported that haplotypes at the Major Histocompatibility Complex (MHC) locus present in some children with autism, or their mothers, might predipose their autistic children to autoimmunity (Burger and Warren, 1998). In two studies, autoantibodies to certain brain tissues and proteins, including myelin basic protein, neurofilament proteins and vascular epithelium were found more often in autistic children compared to controls (Singh et al., 1993; Connolly et al., 1999; Weizman et al., 1982).

Although most autism cases are consistent with the proposed mechanism of oligogenicity and epistasis, a minority have been seen in association with chromosomal abnormalities and with disorders that have specific etiologies. Smalley (1997) stated that approximately 15 to 37% of cases of autism have a comorbid medical condition, including 5 to 14% with a known genetic disorder or chromosomal anomaly. Chromosome anomalies involving almost all human chromosomes have been reported. These include autosomal aneuploidies, sex-chromosome anomalies, deletions, duplications, translocations, ring chromosomes, inversions and marker chromosomes (Gillberg, 1998). Most common are abnormalities of the Prader Willi/Angelman Syndrome region on chromosome 15. Association of autism and a Mendelian condition or genetic syndrome included untreated phenylketonuria, fragile X syndrome, tuberous sclerosis and neurofibromatosis. Recently, Carney et al. (2003) identified mutations in the MECP2 (methyl CpG-binding protein 2) gene in two females with autism who do not have manifestations of Rett syndrome caused in 80% of the cases by mutations in the MECP2 gene.

Different groups are conducting genome scans related to autism or the broader phenotypes of ASDs. This approach appears very promising, because it is both systematic and model free. In addition, it has already been shown to be successful. Thus, positive linkage results have been obtained even by analysing comparatively small study groups. More important, some findings have already been replicated. The most consistent result was obtained for chromosome 7q, but there is also considerable overlap on chromosomes 2q and 16p (Folstein and Rosen-Sheidley, 2001). Considerable progress in identifying chromosomal regions have also been made on chromosome 15 and X. Mutations in two X-linked genes encoding neuroligins NLGN3 and NLGN4 have been identified in siblings with autism spectrum disorders (Jamain et al., 2003). Several lines of evidence support the fact that mutations in neuroligins are involved in autistic disorder. First, the reported mutations cause severe alterations of the predicted protein structure. Second, deletions at Xp22.3 that include NLGN4 have been reported in several autistic children. Third, a mutation in NLGN4 appeared de novo in one affected individual's mother.

### SUMMARY OF THE INVENTION

The present invention now discloses the identification of two human autism susceptibility genes, which can be used for the diagnosis, prevention and treatment of autism, autism spectrum disorders, and autism-associated disorders, as well as for the screening of therapeutically active drugs.

The present invention more particularly discloses the identification of two human autism susceptibility genes, which can be used for the diagnosis, prevention and treatment of autism and related disorders, as well as for the screening of therapeutically active drugs. The invention more specifically discloses certain alleles of the solute carrier family 6 (neurotransmitter transporter, GABA) member 1 gene (SLC6A1) and/or the solute carrier family 6 (neurotransmitter transporter, GABA) member 11 gene (SLC6A11) related to susceptibility to autism and representing novel targets for therapeutic intervention. The present invention relates to particular mutations in the SLC6A1 or SLC6A1 gene and expression products, as well as to diagnostic tools and kits based on these mutations. The invention can be used in the diagnosis of predisposition to, detection, prevention and/or treatment of Asperger syndrome, pervasive developmental disorder, childhood disintegrative disorder, mental retardation, anxiety, depression, attention deficit hyperactivity disorders, speech delay or language impairment, epilepsy, metabolic disorder, immune disorder, bipolar disease and other psychiatric and neurological diseases including schizophrenia.

The invention can be used in the diagnosis of predisposition to or protection from, detection, prevention and/or treatment of autism, an autism spectrum disorder, or an autism-associated disorder, the method comprising detecting in a sample from the subject the presence of an alteration in the SLC6A1 or SLC6A11 gene or polypeptide, the presence of said alteration being indicative of the presence or predisposition to autism, an autism spectrum disorder, or an autism-associated disorder. The presence of said alteration can also be indicative for protecting from autism. In a first preferred embodiment, the method comprises detecting in a sample from the subject the presence of an alteration in the SLC6A1 gene or polypeptide. In a second preferred embodiment, the method comprises detecting in a sample from the subject the presence of an alteration in the SLC6A11 gene or polypeptide.

A particular object of this invention resides in a method of detecting the presence of or predisposition to autism, an autism spectrum disorder, or an autism-associated disorder in a subject, the method comprising detecting the presence of an alteration in the SLC6A1 or SLC6A11 gene locus in a sample from the subject, the presence of said alteration being indicative of the presence of or the predisposition to autism, an autism spectrum disorder, or an autism-associated disorder. In a first preferred embodiment, the method comprises detecting the presence of an alteration in the SLC6A1 gene locus. In a second preferred embodiment, the method comprises detecting the presence of an alteration in the SLC6A 11 gene locus.

An additional particular object of this invention resides in a method of detecting the protection from autism, an autism spectrum disorder, or an autism-associated disorder in a subject, the method comprising detecting the presence of an alteration in the SLC6A1 or SLC6A11 gene locus in a sample from the subject, the presence of said alteration being indicative of the protection from autism, an autism spectrum disorder, or an autism-associated disorder. In a first preferred embodiment, the method comprises detecting the presence of an alteration in the SLC6A1 gene locus. In a second preferred embodiment, the method comprises detecting the presence of an alteration in the SLC6A11 gene locus.

Another particular object of this invention resides in a method of assessing the response of a subject to a treatment of autism, an autism spectrum disorder, or an autism-associated disorder, the method comprising detecting the presence of an alteration in the SLC6A1 or SLC6A11 gene locus in a sample from the subject, the presence of said alteration being indicative of a particular response to said treatment. In a first preferred embodiment, the method comprises detecting the presence of an alteration in the SLC6A1 gene locus. In a second preferred embodiment, the method comprises detecting the presence of an alteration in the SLC6A11 gene locus.

A further particular object of this invention resides in a method of assessing the adverse effect in a subject to a treatment of autism, an autism spectrum disorder, or an autism-associated disorder, the method comprising detecting the presence of an alteration in the SLC6A1 or SLC6A11 gene locus in a sample from the subject, the presence of said alteration being indicative of an adverse effect to said treatment. In a first preferred embodiment, the method comprises detecting the presence of an alteration in the SLC6A1 gene locus. In a second preferred embodiment, the method comprises detecting the presence of an alteration in the SLC6A 11 gene locus.

This invention also relates to a method for preventing autism, an autism spectrum disorder, or an autism-associated disorder in a subject, comprising detecting the presence of an alteration in the SLC6A1 or SLC6A11 gene locus in a sample from the subject, the presence of said alteration being indicative of the predisposition to autism, an autism spectrum disorder, or an autism-associated disorder; and, administering a prophylactic treatment against autism, an autism spectrum disorder, or an autism-associated disorder. In a first preferred embodiment, the method comprises detecting the presence of an alteration in the SLC6A1 gene locus. In a second preferred embodiment, the method comprises detecting the presence of an alteration in the SLC6A11 gene locus.

In a preferred embodiment, said alteration is one or several SNP(s) or a haplotype of SNPs associated with autism.

Preferably, the alteration in the SLC6A1 or SLC6A11 gene locus is determined by performing a hydridization assay, a sequencing assay, a microsequencing assay, or an allele-specific amplification assay.

A particular aspect of this invention resides in compositions of matter comprising primers, probes, and/or oligonucleotides, which are designed to specifically detect at least one SNP or haplotype associated with autism in the genomic region including the SLC6A1 or SLC6A11 gene, or a combination thereof.

The invention also resides in methods of treating autism and/or associated disorders in a subject through a modulation of SLC6A1 and/or SLC6A11 expression or activity. Such treatments use, for instance, SLC6A1 and/or SLC6A11 polypeptides, SLC6A1 and/or SLC6A11 DNA sequences (including antisense sequences and RNAi directed at the SLC6A1 or SLC6A11 gene locus), anti-SLC6A1 and/or anti-SLC6A11 antibodies or drugs that modulate SLC6A1 and/or SLC6A11 expression or activity.

The invention also relates to methods of treating individuals who carry deleterious alleles of the SLC6A1 or SLC6A11 gene, including pre-symptomatic treatment or combined therapy, such as through gene therapy, protein replacement therapy or through the administration of SLC6A1 or SLC6 A11 protein mimetics and/or inhibitors.

A further aspect of this invention resides in the screening of drugs for therapy of autism or associated disorder, based on the modulation of or binding to an allele of SLC6A1 or SLC6A11 gene associated with autism or associated disorder or gene product thereof.

A further aspect of this invention includes antibodies specific of SLC6A1 or SLC6A11 polypeptide fragments and derivatives of such antibodies, hybridomas secreting such antibodies, and diagnostic kits comprising those antibodies. More preferably, said antibodies are specific to a SLC6A1 polypeptide or a fragment thereof comprising an alteration, said alteration modifying the activity of SLC6A 1. Alternatively, said antibodies are specific to a SLC6A 11 polypeptide or a fragment thereof comprising an alteration, said alteration modifying the activity of SLC6A 11.

The invention also concerns a SLC6A1 or SLC6A 11 gene or a fragment thereof comprising an alteration, said alteration modifying the activity of SLC6A 1 or SLC6A 11, respectively. The invention further concerns a SLC6A1 or SLC6A11 polypeptide or a fragment thereof comprising an alteration, said alteration modifying the activity of SLC6A1 or SLC6A11, respectively.

### LEGEND TO THE FIGURES

FIG. 1: High density mapping using Genomic Hybrid Identity Profiling (GenomeHIP).

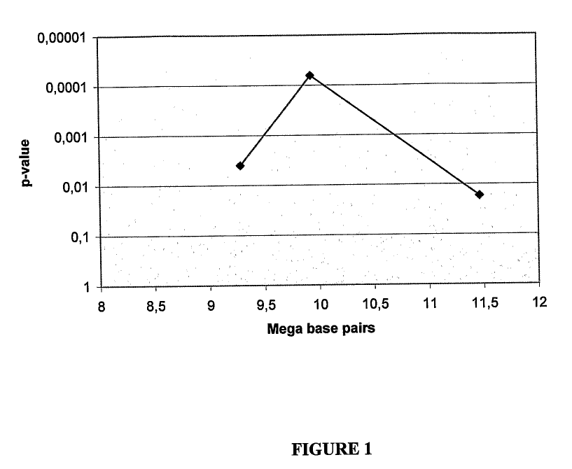

### DETAILED DESCRIPTION OF THE INVENTION

The present invention discloses the identification of SLC6A1 and SLC6A11 as a human autism susceptibility gene. Various nucleic acid samples from 114 families with autism were submitted to a particular GenomeHIP process. This process led to the identification of particular identical-by-descent fragments in said populations that are altered in autistic subjects. By screening of the IBD fragments, we identified the solute carrier family 6 (neurotransmitter transporter, GABA) member 1 gene (SLC6A1) and the solute carrier family 6 (neurotransmitter transporter, GABA) member 11 gene (SLC6A11) on chromosome 3 as candidates for autism and related phenotypes. These genes are indeed present in the critical interval and expresse a functional phenotype consistent with a genetic regulation of autism.

The present invention thus proposes to use SLC6A1 or SLC6A11 gene and corresponding expression products for the diagnosis, prevention and treatment of autism, autism spectrum disorders, and autism-associated disorders, as well as for the screening of therapeutically active drugs.

### DEFINITIONS

Autism and autism spectrum disorders (ASDs): Autism is typically characterized as part of a spectrum of disorders (ASDs) including Asperger syndrome (AS), childhood disintegrative disorder (CDD) and other pervasive developmental disorders (PPD). Autism shall be construed as any condition of impaired social interaction and communication with restricted repetitive and stereotyped patterns of behavior, interests and activities present before the age of 3, to the extent that health may be impaired. AS is distinguished from autistic disorder by the lack of a clinically significant delay in language development in the presence of the impaired social interaction and restricted repetitive behaviors, interests, and activities that characterize the autism-spectrum disorders (ASDs). CDD develops in children who have previously seemed perfectly normal. Typically language, interest in the social environment, and often toileting and self-care abilities are lost, and there may be a general loss of interest in the environment. The child usually comes to look very ‘autistic’, i.e., the clinical presentation (but not the history) is then typical of a child with autism. PPD-NOS (PPD, not otherwise specified) is used to categorize children who do not meet the strict criteria for autism but who come close, either by manifesting atypical autism or by nearly meeting the diagnostic criteria in two or three of the key areas.

Autism-associated disorders, diseases or pathologies include, more specifically, any metabolic and immune disorders, epilepsy, anxiety, depression, attention deficit hyperactivity disorder, speech delay or language impairment, motor incoordination, schizophrenia and bipolar disorder.

The invention may be used in various subjects, particularly human, including adults, children and at the prenatal stage.

Within the context of this invention, the SLC6A1 gene locus designates all SLC6A1 sequences or products in a cell or organism, including SLC6A1 coding sequences, SLC6A1 non-coding sequences (e.g., introns), SLC6A1 regulatory sequences controlling transcription, translation (e.g., promoter, enhancer, terminator, etc.), RNA and/or protein stability, as well as all corresponding expression products, such as SLC6A1 RNAs (e.g., mRNAs) and SLC6A1 polypeptides (e.g., a pre-protein and a mature protein). The SLC6A1 gene locus also comprise surrounding sequences of the SLC6A1 gene which include SNPs that are in linkage disequilibrium with SNPs located in the SLC6A1 gene.

Within the context of this invention, the SLC6A11 gene locus designates all SLC6A11 sequences or products in a cell or organism, including SLC6A11 coding sequences, SLC6A11 non-coding sequences (e.g., introns), SLC6A11 regulatory sequences controlling transcription, translation (e.g., promoter, enhancer, terminator, etc.), RNA and/or protein stability, as well as all corresponding expression products, such as SLC6A11 RNAs (e.g., mRNAs) and SLC6A11 polypeptides (e.g., a pre-protein and a mature protein). The SLC6A11 gene locus also comprise surrounding sequences of the SLC6A11 gene which include SNPs that are in linkage disequilibrium with SNPs located in the SLC6A11 gene.

As used in the present application, the term “SLC6A1 gene” designates the solute carrier family 6 (neurotransmitter, GABA) member 1 gene (SLC6A1) on human chromosome 3, as well as variants, analogs and fragments thereof, including alleles thereof (e.g., germline mutations) which are related to susceptibility to autism and autism-associated disorders. The SLC6A1 gene may also be referred to as GAT1, GABATR, GABATHG.

As used in the present application, the term “SLC6A11 gene” designates the solute carrier family 6 (neurotransmitter, GABA) member 11 gene (SLC6A11) on human chromosome 3, as well as variants, analogs and fragments thereof, including alleles thereof (e.g., germline mutations) which are related to susceptibility to autism and autism-associated disorders. The SLC6A11 gene may also be referred to as GAT3, GAT-3.

The term “gene” shall be construed to include any type of coding nucleic acid, including genomic DNA (gDNA), complementary DNA (cDNA), synthetic or semi-synthetic DNA, as well as any form of corresponding RNA. The term gene particularly includes recombinant nucleic acids encoding SLC6A1 or SLC6A11, i.e., any non naturally occurring nucleic acid molecule created artificially, e.g., by assembling, cutting, ligating or amplifying sequences. A SLC6A1 or SLC6A11 gene is typically double-stranded, although other forms may be contemplated, such as single-stranded. SLC6A1 or SLC6A11 genes may be obtained from various sources and according to various techniques known in the art, such as by screening DNA libraries or by amplification from various natural sources. Recombinant nucleic acids may be prepared by conventional techniques, including chemical synthesis, genetic engineering, enzymatic techniques, or a combination thereof. Suitable SLC6A1 gene sequences may be found on gene banks, such as Unigene Cluster for SLC6A1 (Hs.443874) and Unigene Representative Sequence NM_{—}003042. A particular example of a SLC6A1 gene comprises SEQ ID No: 1. Suitable SLC6A11 gene sequences may be found on gene banks, such as Unigene Cluster for SLC6A11 (Hs.101791) and Unigene Representative Sequence NM_{—}014229. A particular example of a SLC6A11 gene comprises SEQ ID No: 3.

The term “SLC6A1 gene” includes any variant, fragment or analog of SEQ ID No 1 or of any coding sequence as identified above. The term “SLC6A11 gene” includes any variant, fragment or analog of SEQ ID No 3 or of any coding sequence as identified above. Such variants include, for instance, naturally-occurring variants due to allelic variations between individuals (e.g., polymorphisms), mutated alleles related to autism, alternative splicing forms, etc. The term variant also includes SLC6A1 or SLC6A11 gene sequences from other sources or organisms. Variants are preferably substantially homologous to SEQ ID No 1 or 3, i.e., exhibit a nucleotide sequence identity of at least about 65%, typically at least about 75%, preferably at least about 85%, more preferably at least about 95% with SEQ ID No 1 or 3, respectively. Variants and analogs of a SLC6A1 or SLC6A11 gene also include nucleic acid sequences, which hybridize to a sequence as defined above (or a complementary strand thereof) under stringent hybridization conditions.

Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

A fragment of a SLC6A1 or SLC6A11 gene designates any portion of at least about 8 consecutive nucleotides of a sequence as disclosed above, preferably at least about 15, more preferably at least about 20 nucleotides, further preferably of at least 30 nucleotides. Fragments include all possible nucleotide lengths between 8 and 100 nucleotides, preferably between 15 and 100, more preferably between 20 and 100.

A SLC6A1 polypeptide designates any protein or polypeptide encoded by a SLC6A1 gene as disclosed above. The term “polypeptide” refers to any molecule comprising a stretch of amino acids. This term includes molecules of various lengths, such as peptides and proteins. The polypeptide may be modified, such as by glycosylations and/or acetylations and/or chemical reaction or coupling, and may contain one or several non-natural or synthetic amino acids. A specific example of a SLC6A1 polypeptide comprises all or part of SEQ ID No: 2 (NP_{—}003033).

A SLC6A11 polypeptide designates any protein or polypeptide encoded by a SLC6A11 gene as disclosed above. The term “polypeptide” refers to any molecule comprising a stretch of amino acids. This term includes molecules of various lengths, such as peptides and proteins. The polypeptide may be modified, such as by glycosylations and/or acetylations and/or chemical reaction or coupling, and may contain one or several non-natural or synthetic amino acids. A specific example of a SLC6A11 polypeptide comprises all or part of SEQ ID No: 4 (NP_{—}055044).

The terms “response to a treatment” refer to treatment efficacy, including but not limited to ability to metabolize a therapeutic compound, to the ability to convert a pro-drug to an active drug, and to the pharmacokinetics (absorption, distribution, elimination) and the pharmacodynamics (receptor-related) of a drug in an individual.

The terms “adverse effects to a treatment” refer to adverse effects of therapy resulting from extensions of the principal pharmacological action of the drug or to idiosyncratic adverse reactions resulting from an interaction of the drug with unique host factors. “Side effects to a treatment” include, but are not limited to, adverse reactions such as dermatologic, hematologic or hematologic toxicities and further includes gastric and intestinal ulceration, disturbance in platelet function, renal injury, generalized urticaria, bronchoconstriction, hypotension, and shock.

### Diagnosis

The invention now provides diagnosis methods based on a monitoring of the SLC6A1 and/or SLC6A11 gene locus in a subject. Within the context of the present invention, the term “diagnosis” includes the detection, monitoring, dosing, comparison, etc., at various stages, including early, pre-symptomatic stages, and late stages, in adults, children and pre-birth. Diagnosis typically includes the prognosis, the assessment of a predisposition or risk of development, the characterization of a subject to define most appropriate treatment (pharmacogenetics), etc.

The present invention provides diagnostic methods to determine whether an individual is at risk of developing autism, an autism spectrum disorder, or an autism-associated disorder or suffers from autism, an autism spectrum disorder, or an autism-associated disorder resulting from a mutation or a polymorphism in the SLC6A1 and/or SLC6A11 gene locus. The present invention also provides methods to determine whether an individual is likely to respond positively to a therapeutic agent or whether an individual is at risk of developing an adverse side effect to a therapeutic agent.

A particular object of this invention resides in a method of detecting the presence of or predisposition to autism, an autism spectrum disorder, or an autism-associated disorder in a subject, the method comprising detecting in a sample from the subject the presence of an alteration in the SLC6A1 or SLC6A11 gene locus in said sample. The presence of said alteration is indicative of the presence or predisposition to autism, an autism spectrum disorder, or an autism-associated disorder. Optionally, said method comprises a previous step of providing a sample from a subject. Preferably, the presence of an alteration in the SLC6A1 or SLC6A11 gene locus in said sample is detected through the genotyping of a sample.

Another particular object of this invention resides in a method of detecting the protection from autism, an autism spectrum disorder, or an autism-associated disorder in a subject, the method comprising detecting the presence of an alteration in the SLC6A1 or SLC6A11 gene locus in a sample from the subject, the presence of said alteration being indicative of the protection from autism, an autism spectrum disorder, or an autism-associated disorder.

In a preferred embodiment, said alteration is one or several SNP(s) or a haplotype of SNPs associated with autism.

Another particular object of this invention resides in a method of assessing the response of a subject to a treatment of autism, an autism spectrum disorder, or an autism-associated disorder, the method comprising (i) providing a sample from the subject and (ii) detecting the presence of an alteration in the SLC6A1 or SLC6A11 gene locus in said sample.

Another particular object of this invention resides in a method of assessing the response of a subject to a treatment of autism, an autism spectrum disorder, or an autism-associated disorder, the method comprising detecting in a sample from the subject the presence of an alteration in the SLC6A1 or SLC6A11 gene locus in said sample. The presence of said alteration is indicative of a particular response to said treatment. Preferably, the presence of an alteration in the SLC6A1 or SLC6A11 gene locus in said sample is detected through the genotyping of a sample.

A further particular object of this invention resides in a method of assessing the adverse effects of a subject to a treatment of autism, an autism spectrum disorder, or an autism-associated disorder, the method comprising detecting in a sample from the subject the presence of an alteration in the SLC6A1 or SLC6A11 gene locus in said sample. The presence of said alteration is indicative of adverse effects to said treatment. Preferably, the presence of an alteration in the SLC6A1 or SLC6A11 gene locus in said sample is detected through the genotyping of a sample.

In a preferred embodiment, said alteration is one or several SNP(s) or a haplotype of SNPs associated with autism.

In an additional embodiment, the invention concerns a method for preventing autism, an autism spectrum disorder, or an autism-associated disorder in a subject, comprising detecting the presence of an alteration in the SLC6A1 or SLC6A11 gene locus in a sample from the subject, the presence of said alteration being indicative of the predisposition to autism, an autism spectrum disorder, or an autism-associated disorder; and, administering a prophylactic treatment against autism, an autism spectrum disorder, or an autism-associated disorder. Said prophylactic treatment can be a drug administration.

Diagnostics, which analyse and predict response to a treatment or drug, or side effects to a treatment or drug, may be used to determine whether an individual should be treated with a particular treatment drug. For example, if the diagnostic indicates a likelihood that an individual will respond positively to treatment with a particular drug, the drug may be administered to the individual. Conversely, if the diagnostic indicates that an individual is likely to respond negatively to treatment with a particular drug, an alternative course of treatment may be prescribed. A negative response may be defined as either the absence of an efficacious response or the presence of toxic side effects.

Clinical drug trials represent another application for the SLC6A1 or SLC6A11 SNPs. One or more SLC6A 1 or SLC6A11 SNPs indicative of response to a drug or to side effects to a drug may be identified using the methods described above. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

The alteration may be determined at the level of the SLC6A1 or SLC6A11 gDNA, RNA or polypeptide. Optionally, the detection is performed by sequencing all or part of the SLC6A1 or SLC6A11 gene or by selective hybridisation or amplification of all or part of the SLC6A1 or SLC6A11 gene. More preferably a SLC6A1 or SLC6A11 gene specific amplification is carried out before the alteration identification step.

An alteration in the SLC6A1 or SLC6A11 gene locus may be any form of mutation(s), deletion(s), rearrangement(s) and/or insertions in the coding and/or non-coding region of the locus, alone or in various combination(s). Mutations more specifically include point mutations. Deletions may encompass any region of two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion of sequences. The SLC6A1 or SLC6A11 gene locus alteration may result in the creation of stop codons, frameshift mutations, amino acid substitutions, particular RNA splicing or processing, product instability, truncated polypeptide production, etc. The alteration may result in the production of a SLC6A1 or SLC6A11 polypeptide with altered function, stability, targeting or structure. The alteration may also cause a reduction in protein expression or, alternatively, an increase in said production.

In a particular embodiment of the method according to the present invention, the alteration in the SLC6A1 or SLC6A11 gene locus is selected from a point mutation, a deletion and an insertion in the SLC6A1 or SLC6A11 gene or corresponding expression product, more preferably a point mutation and a deletion. The alteration may be determined at the level of the SLC6A1 or SLC6A11 gDNA, RNA or polypeptide.

In any method according to the present invention, one or several SNP in the SLC6A1 or SLC6A11 gene and certain haplotypes comprising SNP in the SLC6A1 or SLC6A11 gene can be used in combination with another SNP or haplotype associated with autism, an autism spectrum disorder, or an autism-associated disorder and located in other gene(s).

In another variant, the method comprises detecting the presence of an altered SLC6A1 or SLC6A1 RNA expression. Altered RNA expression includes the presence of an altered RNA sequence, the presence of an altered RNA splicing or processing, the presence of an altered quantity of RNA, etc. These may be detected by various techniques known in the art, including by sequencing all or part of the SLC6A1 or SLC6A11 RNA or by selective hybridisation or selective amplification of all or part of said RNA, for instance.

In a further variant, the method comprises detecting the presence of an altered SLC6A1 or SLC6A11 polypeptide expression. Altered SLC6A1 or SLC6A11 polypeptide expression includes the presence of an altered polypeptide sequence, the presence of an altered quantity of SLC6A1 or SLC6A11 polypeptide, the presence of an altered tissue distribution, etc. These may be detected by various techniques known in the art, including by sequencing and/or binding to specific ligands (such as antibodies), for instance.

As indicated above, various techniques known in the art may be used to detect or quantify altered SLC6A1 or SLC6A11 gene or RNA expression or sequence, including sequencing, hybridisation, amplification and/or binding to specific ligands (such as antibodies). Other suitable methods include allele-specific oligonucleotide (ASO), allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, heteroduplex analysis, RNase protection, chemical mismatch cleavage, ELISA, radio-immunoassays (RIA) and immuno-enzymatic assays (IEMA).

Some of these approaches (e.g., SSCA and CGGE) are based on a change in electrophoretic mobility of the nucleic acids, as a result of the presence of an altered sequence. According to these techniques, the altered sequence is visualized by a shift in mobility on gels. The fragments may then be sequenced to confirm the alteration.

Some others are based on specific hybridisation between nucleic acids from the subject and a probe specific for wild type or altered SLC6A1 or SLC6A11 gene or RNA. The probe may be in suspension or immobilized on a substrate. The probe is typically labeled to facilitate detection of hybrids.

Some of these approaches are particularly suited for assessing a polypeptide sequence or expression level, such as Northern blot, ELISA and RIA. These latter require the use of a ligand specific for the polypeptide, more preferably of a specific antibody.

In a particular, preferred, embodiment, the method comprises detecting the presence of an altered SLC6A1 or SLC6A11 gene expression profile in a sample from the subject. As indicated above, this can be accomplished more preferably by sequencing, selective hybridisation and/or selective amplification of nucleic acids present in said sample.

### Sequencing

Sequencing can be carried out using techniques well known in the art, using automatic sequencers. The sequencing may be performed on the complete SLC6A1 or SLC6A11 gene or, more preferably, on specific domains thereof, typically those known or suspected to carry deleterious mutations or other alterations.

### Amplification

Amplification is based on the formation of specific hybrids between complementary nucleic acid sequences that serve to initiate nucleic acid reproduction.

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Preferred techniques use allele-specific PCR or PCR-SSCP. Amplification usually requires the use of specific nucleic acid primers, to initiate the reaction.

Nucleic acid primers useful for amplifying sequences from the SLC6A1 or SLC6A11 gene or locus are able to specifically hybridize with a portion of the SLC6A1 or SLC6A11 gene locus that flank a target region of said locus, said target region being altered in certain subjects having autism, an autism spectrum disorder, or an autism-associated disorder.

Primers that can be used to amplify SLC6A1 or SLC6A11 target region comprising SNPs may be designed based on the sequence of Seq Id No 1 or 3 or on the genomic sequence of SLC6A1 or SLC6A11.

Another particular object of this invention resides in a nucleic acid primer useful for amplifying sequences from the SLC6A1 or SLC6A11 gene or locus including surrounding regions. Such primers are preferably complementary to, and hybridize specifically to nucleic acid sequences in the SLC6A1 or SLC6A11 gene locus. Particular primers are able to specifically hybridise with a portion of the SLC6A1 or SLC6A11 gene locus that flank a target region of said locus, said target region being altered in certain subjects having autism, an autism spectrum disorder, or an autism-associated disorder.

The invention also relates to a nucleic acid primer, said primer being complementary to and hybridizing specifically to a portion of a SLC6A1 or SLC6A11 coding sequence (e.g., gene or RNA) altered in certain subjects having autism, an autism spectrum disorder, or an autism-associated disorder. In this regard, particular primers of this invention are specific for altered sequences in a SLC6A1 or SLC6A11 gene or RNA. By using such primers, the detection of an amplification product indicates the presence of an alteration in the SLC6A1 or SLC6A11 gene locus. In contrast, the absence of amplification product indicates that the specific alteration is not present in the sample.

Typical primers of this invention are single-stranded nucleic acid molecules of about 5 to 60 nucleotides in length, more preferably of about 8 to about 25 nucleotides in length. The sequence can be derived directly from the sequence of the SLC6A1 or SLC6A11 gene locus. Perfect complementarity is preferred, to ensure high specificity. However, certain mismatch may be tolerated.

The invention also concerns the use of a nucleic acid primer or a pair of nucleic acid primers as described above in a method of detecting the presence of or predisposition to autism, an autism spectrum disorder, or an autism-associated disorder in a subject or in a method of assessing the response of a subject to a treatment of autism, an autism spectrum disorder, or an autism-associated disorder.

### Selective Hybridization

Hybridization detection methods are based on the formation of specific hybrids between complementary nucleic acid sequences that serve to detect nucleic acid sequence alteration(s).

A particular detection technique involves the use of a nucleic acid probe specific for wild type or altered SLC6A1 or SLC6A11 gene or RNA, followed by the detection of the presence of a hybrid. The probe may be in suspension or immobilized on a substrate or support (as in nucleic acid array or chips technologies). The probe is typically labeled to facilitate detection of hybrids.

In this regard, a particular embodiment of this invention comprises contacting the sample from the subject with a nucleic acid probe specific for an altered SLC6A1 or SLC6A11 gene locus, and assessing the formation of an hybrid. In a particular, preferred embodiment, the method comprises contacting simultaneously the sample with a set of probes that are specific, respectively, for wild type SLC6A1 or SLC6A11 gene locus and for various altered forms thereof. In this embodiment, it is possible to detect directly the presence of various forms of alterations in the SLC6A1 or SLC6A11 gene locus in the sample. Also, various samples from various subjects may be treated in parallel.

Within the context of this invention, a probe refers to a polynucleotide sequence which is complementary to and capable of specific hybridisation with a (target portion of a) SLC6A1 or SLC6A11 gene or RNA, and which is suitable for detecting polynucleotide polymorphisms associated with SLC6A1 or SLC6A11 alleles which predispose to or are associated with autism, an autism spectrum disorder, or an autism-associated disorder. Probes are preferably perfectly complementary to the SLC6A1 or SLC6A11 gene, RNA, or target portion thereof. Probes typically comprise single-stranded nucleic acids of between 8 to 1000 nucleotides in length, for instance of between 10 and 800, more preferably of between 15 and 700, typically of between 20 and 500. It should be understood that longer probes may be used as well. A preferred probe of this invention is a single stranded nucleic acid molecule of between 8 to 500 nucleotides in length, which can specifically hybridise to a region of a SLC6A1 or SLC6A11 gene or RNA that carries an alteration.

A specific embodiment of this invention is a nucleic acid probe specific for an altered (e.g., a mutated) SLC6A1 or SLC6A11 gene or RNA, i.e., a nucleic acid probe that specifically hybridises to said altered SLC6A1 or SLC6A11 gene or RNA and essentially does not hybridise to a SLC6A1 or SLC6A11 gene or RNA lacking said alteration. Specificity indicates that hybridisation to the target sequence generates a specific signal which can be distinguished from the signal generated through non-specific hybridisation. Perfectly complementary sequences are preferred to design probes according to this invention. It should be understood, however, that a certain degree of mismatch may be tolerated, as long as the specific signal may be distinguished from non-specific hybridisation.

The sequence of the probes can be derived from the sequences of the SLC6A1 or SLC6A11 gene and RNA as provided in the present application. Nucleotide substitutions may be performed, as well as chemical modifications of the probe. Such chemical modifications may be accomplished to increase the stability of hybrids (e.g., intercalating groups) or to label the probe. Typical examples of labels include, without limitation, radioactivity, fluorescence, luminescence, enzymatic labeling, etc.

The invention also concerns the use of a nucleic acid probe as described above in a method of detecting the presence of or predisposition to autism, an autism spectrum disorder, or an autism-associated disorder in a subject or in a method of assessing the response of a subject to a treatment of autism, an autism spectrum disorder, or an autism-associated disorder.

### Specific Ligand Binding

As indicated above, alteration in the SLC6A1 or SLC6A11 gene locus may also be detected by screening for alteration(s) in SLC6A1 or SLC6A11 polypeptide sequence or expression levels, respectively. In this regard, a specific embodiment of this invention comprises contacting the sample with a ligand specific for a SLC6A1 or SLC6A11 polypeptide and determining the formation of a complex.

Different types of ligands may be used, such as specific antibodies. In a specific embodiment, the sample is contacted with an antibody specific for a SLC6A1 or SLC6A11 polypeptide and the formation of an immune complex is determined. Various methods for detecting an immune complex can be used, such as ELISA, radioimmunoassays (RIA) and immuno-enzymatic assays (IEMA).

Within the context of this invention, an antibody designates a polyclonal antibody, a monoclonal antibody, as well as fragments or derivatives thereof having substantially the same antigen specificity. Fragments include Fab, Fab'2, CDR regions, etc. Derivatives include single-chain antibodies, humanized antibodies, poly-functional antibodies, etc.

An antibody specific for a SLC6A1 or SLC6A11 polypeptide designates an antibody that selectively binds a SLC6A1 or SLC6A11 polypeptide, respectively, namely, an antibody raised against a SLC6A1 or SLC6A11 polypeptide, respectively, or an epitope-containing fragment thereof. Although non-specific binding towards other antigens may occur, binding to the target SLC6A1 or SLC6A1 polypeptide occurs with a higher affinity and can be reliably discriminated from non-specific binding.

In a specific embodiment, the method comprises contacting a sample from the subject with (a support coated with) an antibody specific for an altered form of a SLC6A1 or SLC6A11 polypeptide, and determining the presence of an immune complex. In a particular embodiment, the sample may be contacted simultaneously, or in parallel, or sequentially, with various (supports coated with) antibodies specific for different forms of a SLC6A1 or SLC6A11 polypeptide, such as a wild type and various altered forms thereof.

The invention also concerns the use of a ligand, preferably an antibody, a fragment or a derivative thereof as described above, in a method of detecting the presence of or predisposition to autism, an autism spectrum disorder, or an autism-associated disorder in a subject or in a method of assessing the response of a subject to a treatment of autism, an autism spectrum disorder, or an autism-associated disorder.

The invention also relates to a diagnostic kit comprising products and reagents for detecting in a sample from a subject the presence of an alteration in the SLC6A1 or SLC6A11 gene or polypeptide, in the SLC6A1 or SLC6A11 gene or polypeptide expression, and/or in SLC6A1 or SLC6A11 activity. Said diagnostic kit according to the present invention comprises any primer, any pair of primers, any nucleic acid probe and/or any ligand, preferably antibody, described in the present invention. Said diagnostic kit according to the present invention can further comprise reagents and/or protocols for performing a hybridization, amplification or antigen-antibody immune reaction.

The diagnosis methods can be performed in vitro, ex vivo or in vivo, preferably in vitro or ex vivo. They use a sample from the subject, to assess the status of the SLC6A1 or SLC6A11 gene locus. The sample may be any biological sample derived from a subject, which contains nucleic acids or polypeptides. Examples of such samples include fluids, tissues, cell samples, organs, biopsies, etc. Most preferred samples are blood, plasma, saliva, urine, seminal fluid, etc. Pre-natal diagnosis may also be performed by testing fetal cells or placental cells, for instance. The sample may be collected according to conventional techniques and used directly for diagnosis or stored. The sample may be treated prior to performing the method, in order to render or improve availability of nucleic acids or polypeptides for testing. Treatments include, for instant, lysis (e.g., mechanical, physical, chemical, etc.), centrifugation, etc. Also, the nucleic acids and/or polypeptides may be pre-purified or enriched by conventional techniques, and/or reduced in complexity. Nucleic acids and polypeptides may also be treated with enzymes or other chemical or physical treatments to produce fragments thereof. Considering the high sensitivity of the claimed methods, very few amounts of sample are sufficient to perform the assay.

As indicated, the sample is preferably contacted with reagents such as probes, primers or ligands in order to assess the presence of an altered SLC6A1 or SLC6A11 gene locus. Contacting may be performed in any suitable device, such as a plate, tube, well, glass, etc. In specific embodiments, the contacting is performed on a substrate coated with the reagent, such as a nucleic acid array or a specific ligand array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids or polypeptides of the sample.

The finding of an altered SLC6A1 or SLC6A11 polypeptide, RNA or DNA in the sample is indicative of the presence of an altered SLC6A1 or SLC6A11 gene locus in the subject, which can be correlated to the presence, predisposition or stage of progression of autism, an autism spectrum disorder, or an autism-associated disorder. For example, an individual having a germ line SLC6A1 or SLC6A11 mutation has an increased risk of developing autism, an autism spectrum disorder, or an autism-associated disorder. The determination of the presence of an altered SLC6A1 or SLC6A11 gene locus in a subject also allows the design of appropriate therapeutic intervention, which is more effective and customized. Also, this determination at the pre-symptomatic level allows a preventive regimen to be applied.

### Linkage Disequilibirum

Once a first SNP has been identified in a genomic region of interest, more particularly in SLC6A1 or SLC6A11 gene locus, the practitioner of ordinary skill in the art can easily identify additional SNPs in linkage disequilibrium with this first SNP. Indeed, any SNP in linkage disequilibrium with a first SNP associated with autism or an associated disorder will be associated with this trait. Therefore, once the association has been demonstrated between a given SNP and autism or an associated disorder, the discovery of additional SNPs associated with this trait can be of great interest in order to increase the density of SNPs in this particular region.

Identification of additional SNPs in linkage disequilibrium with a given SNP involves: (a) amplifying a fragment from the genomic region comprising or surrounding a first SNP from a plurality of individuals; (b) identifying of second SNPs in the genomic region harboring or surrounding said first SNP; (c) conducting a linkage disequilibrium analysis between said first SNP and second SNPs; and (d) selecting said second SNPs as being in linkage disequilibrium with said first marker. Subcombinations comprising steps (b) and (c) are also contemplated.

Methods to identify SNPs and to conduct linkage disequilibrium analysis can be carried out by the skilled person without undue experimentation by using well-known methods.

These SNPs in linkage disequilibrium can also be used in the methods according to the present invention, and more particularly in the diagnostic methods according to the present invention.

For example, a linkage locus of Crohn's disease has been mapped to a large region spanning 18cM on chromosome 5q31 (Rioux et al., 2000 and 2001). Using dense maps of microsatellite markers and SNPs across the entire region, strong evidence of linkage disequilibrium (LD) was found. Having found evidence of LD, the authors developed an ultra-high-density SNP map and studied a denser collection of markers selected from this map. Multilocus analyses defined a single common risk haplotype characterised by multiple SNPs that were each independently associated using TDT. These SNPs were unique to the risk haplotype and essentially identical in their information content by virtue of being in nearly complete LD with one another. The equivalent properties of these SNPs make it impossible to identify the causal mutation within this region on the basis of genetic evidence alone.

### Causal Mutation

Mutations in the SLC6A1 or SLC6A11 gene which are responsible for autism or an associated disorder may be identified by comparing the sequences of the SLC6A1 or SLC6A11 gene, respectively, from patients presenting autism or an associated disorder and control individuals. Based on the identified association of SNPs of SLC6A1 or SLC6A11 and autism or an associated disorder, the identified locus can be scanned for mutations. In a preferred embodiment, functional regions such as exons and splice sites, promoters and other regulatory regions of the SLC6A1 or SLC6A11 gene are scanned for mutations. Preferably, patients presenting autism or an associated disorder carry the mutation shown to be associated with autism or an associated disorder and controls individuals do not carry the mutation or allele associated with autism or an associated disorder. It might also be possible that patients presenting autism or an associated disorder carry the mutation shown to be associated with autism or an associated disorder with a higher frequency than controls individuals.

The method used to detect such mutations generally comprises the following steps: amplification of a region of the SLC6A1 or SLC6A11 gene comprising a SNP or a group of SNPs associated with autism or an associated disorder from DNA samples of the SLC6A1 or SLC6A 11 gene, respectively, from patients presenting autism or an associated disorder and control individuals; sequencing of the amplified region; comparison of DNA sequences of the SLC6A1 or SLC6A11 gene, respectively, from patients presenting autism or an associated disorder and control individuals; determination of mutations specific to patients presenting autism or an associated disorder.

Therefore, identification of a causal mutation in the SLC6A1 or SLC6A11 gene can be carried out by the skilled person without undue experimentation by using well-known methods.

For example, the causal mutations have been identified in the following examples by using routine methods.

Hugot et al. (2001) applied a positional cloning strategy to identify gene variants with susceptibly to Crohn's disease in a region of chromosome 16 previously found to be linked to susceptibility to Crohn's disease. To refine the location of the potential susceptibility locus 26 microsatellite markers were genotyped and tested for association to Crohn's disease using the transmission disequilibrium test. A borderline significant association was found between one allele of the microsatellite marker D16S136. Eleven additional SNPs were selected from surrounding regions and several SNPs showed significant association. SNP5-8 from this region were found to be present in a single exon of the NOD2/CARD15 gene and shown to be non-synonymous variants. This prompted the authors to sequence the complete coding sequence of this gene in 50 CD patients. Two additional non-synonymous mutations (SNP12 and SNP13) were found. SNP13 was most significant associated (p=6×10−6) using the pedigree transmission disequilibrium test. In another independent study, the same variant was found also by sequencing the coding region of this gene from 12 affected individuals compared to 4 controls (Ogura et al., 2001). The rare allele of SNP13 corresponded to a 1-bp insertion predicted to truncate the NOD2/CARD15 protein. This allele was also present in normal healthy individuals, albeit with significantly lower frequency as compared to the controls.

Similarly, Lesage et al. (2002) performed a mutational analyses of CARD15 in 453 patients with CD, including 166 sporadic and 287 familial cases, 159 patients with ulcerative colitis (UC), and 103 healthy control subjects by systematic sequencing of the coding region. Of 67 sequence variations identified, 9 had an allele frequency >5% in patients with CD. Six of them were considered to be polymorphisms, and three (SNP12-R702W, SNP8-G908R, and SNP13-1007 fs) were confirmed to be independently associated with susceptibility to CD. Also considered as potential disease-causing mutations (DCMs) were 27 rare additional mutations. The three main variants (R702W, G908R, and 1007 fs) represented 32%, 18%, and 31%, respectively, of the total CD mutations, whereas the total of the 27 rare mutations represented 19% of DCMs. Altogether, 93% of the mutations were located in the distal third of the gene. No mutations were found to be associated with UC. In contrast, 50% of patients with CD carried at least one DCM, including 17% who had a double mutation.

### Drug Screening

The present invention also provides novel targets and methods for the screening of drug candidates or leads. The methods include binding assays and/or functional assays, and may be performed in vitro, in cell systems, in animals, etc.

A particular object of this invention resides in a method of selecting biologically active compounds, said method comprising contacting in vitro a test compound with a SLC6A1 or SLC6A11 gene or polypeptide according to the present invention and determining the ability of said test compound to bind said SLC6A1 or SLC6A11 gene or polypeptide, respectively. Binding to said gene or polypeptide provides an indication as to the ability of the compound to modulate the activity of said target, and thus to affect a pathway leading to autism, an autism spectrum disorder, or an autism-associated disorder in a subject. In a preferred embodiment, the method comprises contacting in vitro a test compound with a SLC6A1 or SLC6A11 polypeptide or a fragment thereof according to the present invention and determining the ability of said test compound to bind said SLC6A1 or SLC6A11 polypeptide or fragment, respectively. The fragment preferably comprises a binding site of the SLC6A1 or SLC6A11 polypeptide. Preferably, said SLC6A1 gene or polypeptide or a fragment thereof is an altered or mutated SLC6A1 gene or polypeptide or a fragment thereof comprising the alteration or mutation. Preferably, said SLC6A 11 gene or polypeptide or a fragment thereof is an altered or mutated SLC6A11 gene or polypeptide or a fragment thereof comprising the alteration or mutation.

A particular object of this invention resides in a method of selecting compounds active on autism, autism spectrum disorders, and autism-associated disorders, said method comprising contacting in vitro a test compound with a SLC6A1 or SLC6A11 polypeptide according to the present invention or binding site-containing fragment thereof and determining the ability of said test compound to bind said SLC6A1 or SLC6A1 polypeptide or fragment thereof, respectively. Preferably, said SLC6A1 polypeptide or a fragment thereof is an altered or mutated SLC6A1 polypeptide or a fragment thereof comprising the alteration or mutation. Preferably, said SLC6A11 polypeptide or a fragment thereof is an altered or mutated SLC6A11 polypeptide or a fragment thereof comprising the alteration or mutation.

In a further particular embodiment, the method comprises contacting a recombinant host cell expressing a SLC6A1 or SLC6A11 polypeptide according to the present invention with a test compound, and determining the ability of said test compound to bind said SLC6A1 or SLC6A 1, respectively, and to modulate the activity of SLC6A1 or SLC6A 11 polypeptide, respectively. Preferably, said SLC6A1 polypeptide or a fragment thereof is an altered or mutated SLC6A1 polypeptide or a fragment thereof comprising the alteration or mutation. Preferably, said SLC6A 1 polypeptide or a fragment thereof is an altered or mutated SLC6A11 polypeptide or a fragment thereof comprising the alteration or mutation.

The determination of binding may be performed by various techniques, such as by labeling of the test compound, by competition with a labeled reference ligand, etc.

A further object of this invention resides in a method of selecting biologically active compounds, said method comprising contacting in vitro a test compound with a SLC6A1 or SLC6A11 polypeptide according to the present invention and determining the ability of said test compound to modulate the activity of said SLC6A1 or SLC6A11 polypeptide, respectively. Preferably, said SLC6A1 polypeptide or a fragment thereof is an altered or mutated SLC6A1 polypeptide or a fragment thereof comprising the alteration or mutation. Preferably, said SLC6A11 polypeptide or a fragment thereof is an altered or mutated SLC6A11 polypeptide or a fragment thereof comprising the alteration or mutation.

A further object of this invention resides in a method of selecting biologically active compounds, said method comprising contacting in vitro a test compound with a SLC6A1 or SLC6A11 gene according to the present invention and determining the ability of said test compound to modulate the expression of said SLC6A1 or SLC6A11 gene, respectively. Preferably, said SLC6A1 gene or a fragment thereof is an altered or mutated SLC6A1 gene or a fragment thereof comprising the alteration or mutation. Preferably, said SLC6A11 gene or a fragment thereof is an altered or mutated SLC6A11 gene or a fragment thereof comprising the alteration or mutation.

In an other embodiment, this invention relates to a method of screening, selecting or identifying active compounds, particularly compounds active on autism, an autism spectrum disorder, or an autism-associated disorder, the method comprising contacting a test compound with a recombinant host cell comprising a reporter construct, said reporter construct comprising a reporter gene under the control of a SLC6A1 or SLC6A11 gene promoter, and selecting the test compounds that modulate (e.g. stimulate or reduce) expression of the reporter gene. Preferably, said SLC6A1 gene promoter or a fragment thereof is an altered or mutated SLC6A1 gene promoter or a fragment thereof comprising the alteration or mutation. Preferably, said SLC6A11 gene promoter or a fragment thereof is an altered or mutated SLC6A 1 gene promoter or a fragment thereof comprising the alteration or mutation.

In a particular embodiment of the methods of screening, the modulation is an inhibition. In another particular embodiment of the methods of screening, the modulation is an activation.

The above screening assays may be performed in any suitable device, such as plates, tubes, dishes, flasks, etc. Typically, the assay is performed in multi-wells plates. Several test compounds can be assayed in parallel. Furthermore, the test compound may be of various origin, nature and composition. It may be any organic or inorganic substance, such as a lipid, peptide, polypeptide, nucleic acid, small molecule, etc., in isolated or in mixture with other substances. The compounds may be all or part of a combinatorial library of products, for instance.

### Pharmaceutical Compositions, Therapy

A further object of this invention is a pharmaceutical composition comprising (i) a SLC6A1 or SLC6A11 polypeptide or a fragment thereof, a nucleic acid encoding a SLC6A1 or SLC6A11 polypeptide or a fragment thereof, a vector or a recombinant host cell as described above and (ii) a pharmaceutically acceptable carrier or vehicle.

The invention also relates to a method of treating or preventing autism, an autism spectrum disorder, or an autism-associated disorder in a subject, the method comprising administering to said subject a functional (e.g., wild-type) SLC6A1 or SLC6A11 polypeptide or a nucleic acid encoding the same.

An other embodiment of this invention resides in a method of treating or preventing autism, an autism spectrum disorder, or an autism-associated disorder in a subject, the method comprising administering to said subject a compound that modulates, preferably that activates or mimics, expression or activity of a SLC6A1 or SLC6A11 gene or protein according to the present invention. Said compound can be an agonist or an antagonist of SLC6A1 or SLC6A11, an antisense or a RNAi of SLC6A1 or SLC6A11, an antibody or a fragment or a derivative thereof specific to a SLC6A1 or SLC6A11 polypeptide according to the present invention. In a particular embodiment of the method, the modulation is an inhibition. In another particular embodiment of the method, the modulation is an activation.

The invention also relates, generally, to the use of a functional SLC6A1 or SLC6A11 polypeptide, a nucleic acid encoding the same, or a compound that modulates expression or activity of a SLC6A1 or SLC6A11 gene or protein according to the present invention, in the manufacture of a pharmaceutical composition for treating or preventing autism, an autism spectrum disorder, or an autism-associated disorder in a subject. Said compound can be an agonist or an antagonist of SLC6A1 or SLC6A11, an antisense or a RNAi of SLC6A1 or SLC6A11, an antibody or a fragment or a derivative thereof specific to a SLC6A1 or SLC6A11 polypeptide according to the present invention. In a particular embodiment of the method, the modulation is an inhibition. In another particular embodiment of the method, the modulation is an activation.

The present invention demonstrates the correlation between autism, autism spectrum disorders, and autism-associated disorders and the SLC6A1 or SLC6A11 gene locus. The invention thus provides a novel target of therapeutic intervention. Various approaches can be contemplated to restore or modulate the SLC6A1 or SLC6A11 activity or function in a subject, particularly those carrying an altered SLC6A1 or SLC6A11 gene locus. Supplying wild-type function to such subjects is expected to suppress phenotypic expression of autism, autism spectrum disorders, and autism-associated disorders in a pathological cell or organism. The supply of such function can be accomplished through gene or protein therapy, or by administering compounds that modulate or mimic SLC6A1 or SLC6A11 polypeptide activity (e.g., agonists as identified in the above screening assays).

The wild-type SLC6A1 or SLC6A11 gene or a functional part thereof may be introduced into the cells of the subject in need thereof using a vector as described above. The vector may be a viral vector or a plasmid. The gene may also be introduced as naked DNA. The gene may be provided so as to integrate into the genome of the recipient host cells, or to remain extra-chromosomal. Integration may occur randomly or at precisely defined sites, such as through homologous recombination. In particular, a functional copy of the SLC6A1 or SLC6A11 gene may be inserted in replacement of an altered version in a cell, through homologous recombination. Further techniques include gene gun, liposome-mediated transfection, cationic lipid-mediated transfection, etc. Gene therapy may be accomplished by direct gene injection, or by administering ex vivo prepared genetically modified cells expressing a functional SLC6A1 or SLC6A11 polypeptide.

Other molecules with SLC6A1 or SLC6A11 activity (e.g., peptides, drugs, SLC6A1 or SLC6A11 agonists, or organic compounds) may also be used to restore functional SLC6A1 or SLC6A11 activity in a subject or to suppress the deleterious phenotype in a cell.

Restoration of functional SLC6A1 or SLC6A11 gene function in a cell may be used to prevent the development of autism, an autism spectrum disorder, or an autism-associated disorder or to reduce progression of said diseases. Such a treatment may suppress the autism-associated phenotype of a cell, particularly those cells carrying a deleterious allele.

Further aspects and advantages of the present invention will be disclosed in the following experimental section, which should be regarded as illustrative and not limiting the scope of the present application.

### Gene, Vectors, Recombinant Cells and Polypeptides

A further aspect of this invention resides in novel products for use in diagnosis, therapy or screening. These products comprise nucleic acid molecules encoding a SLC6A1 or SLC6A11 polypeptide or a fragment thereof, vectors comprising the same, recombinant host cells and expressed polypeptides.

More particularly, the invention concerns an altered or mutated SLC6A1 or SLC6A11 gene or a fragment thereof comprising said alteration or mutation. The invention also concerns nucleic acid molecules encoding an altered or mutated SLC6A1 or SLC6A11 polypeptide or a fragment thereof comprising said alteration or mutation. Said alteration or mutation modifies the SLC6A1 or SLC6A11 activity. The modified activity can be increased or decreased. The invention further concerns a vector comprising an altered or mutated SLC6A1 or SLC6A11 gene or a fragment thereof comprising said alteration or mutation or a nucleic acid molecule encoding an altered or mutated SLC6A1 or SLC6A11 polypeptide or a fragment thereof comprising said alteration or mutation, recombinant host cells and expressed polypeptides.

A further object of this invention is a vector comprising a nucleic acid encoding a SLC6A1 or SLC6A11 polypeptide according to the present invention. The vector may be a cloning vector or, more preferably, an expression vector, i.e., a vector comprising regulatory sequences causing expression of a SLC6A1 or SLC6A11 polypeptide from said vector in a competent host cell.

These vectors can be used to express a SLC6A1 or SLC6A11 polypeptide in vitro, ex vivo or in vivo, to create transgenic or “Knock Out” non-human animals, to amplify the nucleic acids, to express antisense RNAs, etc.

The vectors of this invention typically comprise a SLC6A1 or SLC6A11 coding sequence according to the present invention operably linked to regulatory sequences, e.g., a promoter, a polyA, etc. The term “operably linked” indicates that the coding and regulatory sequences are functionally associated so that the regulatory sequences cause expression (e.g., transcription) of the coding sequences. The vectors may further comprise one or several origins of replication and/or selectable markers. The promoter region may be homologous or heterologous with respect to the coding sequence, and may provide for ubiquitous, constitutive, regulated and/or tissue specific expression, in any appropriate host cell, including for in vivo use. Examples of promoters include bacterial promoters (T7, pTAC, Trp promoter, etc.), viral promoters (LTR, TK, CMV-IE, etc.), mammalian gene promoters (albumin, PGK, etc), and the like.

The vector may be a plasmid, a virus, a cosmid, a phage, a BAC, a YAC, etc. Plasmid vectors may be prepared from commercially available vectors such as pBluescript, pUC, pBR, etc. Viral vectors may be produced from baculoviruses, retroviruses, adenoviruses, AAVs, etc., according to recombinant DNA techniques known in the art.

In this regard, a particular object of this invention resides in a recombinant virus encoding a SLC6A1 or SLC6A1 polypeptide as defined above. The recombinant virus is preferably replication-defective, even more preferably selected from E1- and/or E4-defective adenoviruses, Gag-, pol- and/or env-defective retroviruses and Rep- and/or Cap-defective AAVs. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+cells, 293 cells, etc. Detailed protocols for producing such replication-defective recombinant viruses may be found for instance in WO95/14785, WO96/22378, U.S. Pat. No. 5,882,877, U.S. Pat. No. 6,013,516, U.S. Pat. No. 4,861,719, U.S. Pat. No. 5,278,056 and WO94/19478.

A further object of the present invention resides in a recombinant host cell comprising a recombinant SLC6A1 or SLC6A11 gene or a vector as defined above. Suitable host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from fibroblasts, embryonic cells, epithelial cells, nervous cells, adipocytes, etc.).

The present invention also relates to a method for producing a recombinant host cell expressing a SLC6A1 or SLC6A 11 polypeptide according to the present invention, said method comprising (i) introducing in vitro or ex vivo into a competent host cell a recombinant nucleic acid or a vector as described above, (ii) culturing in vitro or ex vivo the recombinant host cells obtained and (iii), optionally, selecting the cells which express the SLC6A1 or SLC6A11 polypeptide.

Such recombinant host cells can be used for the production of SLC6A1 or SLC6A11 polypeptides, as well as for screening of active molecules, as described below. Such cells may also be used as a model system to study autism. These cells can be maintained in suitable culture media, such as DMEM, RPMI, HAM, etc., in any appropriate culture device (plate, flask, dish, tube, pouch, etc.).

### EXAMPLES

### 1. GenomeHIP Platform to Identify the Chromosome 3 Susceptibility Genes

The GenomeHIP platform was applied to allow rapid identification of an autism susceptibility gene.

Briefly, the technology consists of forming pairs from the DNA of related individuals. Each DNA is marked with a specific label allowing its identification. Hybrids are then formed between the two DNAs. A particular process (WO00/53802) is then applied that selects all fragments identical-by-descent (IBD) from the two DNAs in a multi step procedure. The remaining IBD enriched DNA is then scored against a BAC clone derived DNA microarray that allows the positioning of the IBD fraction on a chromosome.

The application of this process over many different families results in a matrix of IBD fractions for each pair from each family. Statistical analyses then calculate the minimal IBD regions that are shared between all families tested. Significant results (p-values) are evidence for linkage of the positive region with the trait of interest (here autism). The linked interval can be delimited by the two most distant clones showing significant p-values.

In the present study, 114 families from the United States (114 independent sib-pairs) concordant for strict autism (as defined by ADI-R) were submitted to the GenomeHIP process. The resulting IBD enriched DNA fractions were then labeled with Cy5 fluorescent dyes and hybridised against a DNA array consisting of 2263 BAC clones covering the whole human genome with an average spacing of 1.2 Mega base pairs. Non-selected DNA labeled with Cy3 was used to normalize the signal values and compute ratios for each clone. Clustering of the ratio results was then performed to determine the IBD status for each clone and pair.

By applying this procedure, a BAC clone was identified (FE0DBACA17ZG05v) which showed suggestive evidence for linkage to autism (p=6.4e-05). The p-value 2.4e-04 corresponding to the significance level for suggestive linkage was used as a significance level for whole genome screens as proposed by Kruglyak and Lander (1995). The linkage region was spanning approximately 2.18 megabases in the region on chromosome 3 (bases 9283670 to 11464577) as defined by the clones proximal and distal of the BAC clone showing significant evidence for linkage.

Table 1: Linkage results for chromosome 3 in the SLC6A1 and SLC6A11 locus: Indicated is the region corresponding to the BAC clone with evidence for linkage. The start and stop positions of the clones correspond to their genomic locations based on NCBI Build34 with respect to the start of the chromosome (p-ter).

TABLE 1

| Human | | | | N informative | |
|---|---|---|---|---|---|
| chromosome | Clone | Start | Stop | pairs | p-value |
| 3 | FE0DBACA2ZH12v | 9124735 | 9283670 | 81 | 0.004 |
| 3 | FE0DBACA17ZG05v | 9721553 | 9931071 | 104 | 6.4e−05 |
| 3 | FE0DBACA18ZE05v | 11464577 | 11591404 | 77 | 0.017 |

### 2. Identification of Autism Susceptibility Genes on Chromosome 3

By screening the aforementioned 2.18 Megabases in the linked chromosomal region, we identified the solute carrier family 6 (neurotransmitter transporter, GABA), member 1 gene and the solute carrier family 6 (neurotransmitter transporter, GABA), member 11 gene as candidates for autism and related phenotypes. These genes are indeed present in the critical interval, with evidence for linkage delimited by the clones outlined above.

The SLC6A1 gene encodes a predicted 599-amino acid polypeptide for NP_{—}003033 (mRNA NM_{—}003042, 4493 bp) and spreads over 46.5 kb of genomic sequence. The protein encoded by this gene is a member of the sodium:neurotransmitter symporter (SNF) family that transports gamma-aminobutyric acid (GABA) and terminates the action of GABA by its high affinity sodium-dependent reuptake into presynaptic terminals. This protein is the target of psychomotor stimulants such as amphetamines or cocaine.

The SLC6A11 gene encodes a predicted 632-amino acid polypeptide for NP_{—}055044 (mRNA NM_{—}014229, 1991 bp) and spreads over 122.2 kb of genomic sequence. The protein encoded by this gene is a member of the sodium:neurotransmitter symporter (SNF) family that transports gamma-aminobutyric acid (GABA), a major inhibitory neurotransmitter. GABAergic neurotransmission is terminated by the uptake of GABA into the presynaptic terminal and the surrounding astroglial cells by sodium-dependent transporters, such as SLC6A11.

It has been hypothesized that the severe disruptions observed in autism may be linked to GABAergic inhibition, resulting in excessive stimulation of glutamate specialized neurons and loss of sensory gating (Hussman, 2001).

In a hypoglutamatergic rodent model, certain behaviors that might have relevance for the cognitive impairments seen in autism were observed (Nilsson et al., 2001).

Reductions in glutamic acid decarboxylase 65 and 67 kDa levels may account for reported increases of glutamate in blood and platelets of autistic subjects (Fatemi et al., 2002). Glutamic acid decarboxylase deficiency may be due to or associated with abnormalities in levels of glutamate/gamma amino butyric acid, or transporter/receptor density in autistic brain. Furthermore, a decrease of glutamate receptor density has been observed in the cerebellum of autistic patients (Purcell et al., 2001).

Taken together, the linkage results provided in the present application, identifying the human SLC6A1 and SLC6A11 genes in the critical interval of genetic alterations linked to autism on chromosome 3, with its involvement in GABA transport, we conclude that alterations (e.g., mutations and/or polymorphisms) in the SLC6A1 and/or SLC6A11 gene or its regulatory sequences may contribute to the development of human autism and represent a novel target for diagnosis or therapeutic intervention.

### REFERENCES

Asperger (1944) Die autistischen Psychopathen im Kindesalter. Archiv für Psychiatrie und Nervenkrankheiten, 2:217-250.
Bailey A, Le Couteur A, Gottesman I, et al. (1995) Autism as a strongly genetic disorder: evidence from a British twin study. Psychol Med, 25:63-77.
Bailey A, Phillips W, Rutter M (1996) Autism: towards an integration of clinical, genetic, neuropsychological, and neurobiological perspectives. J Child Psychol Psychiatry 37(1):89-126.
Baird G, Charman T, Baron-Cohen S et al. (2000) A screening instrument for autism at 18 months of age: a 6-year follow-up study. J Am Acad Child Adolesc Psychiatry, 39(6):694-702.
Burger R and Warren R (1998) Possible immunogenetic basis for autism. Ment Retard Dev Disabil Res Rev, 4:137-141.
Carney R M, Wolpert C M, Ravan S A et al. (2003) Identification of MeCP2 mutations in a series of females with autistic disorder. Pediatr Neurol, 28(3):205-211.
Chakrabarti S and Fombonne E (2001) Pervasive developmental disorders in preschool children. JAMA, 285(24):3093-9
Comi A M, Zimmerman A W, Frye V H et al. (1999) Familial clustering of autoimmune disorders and evaluation of medical risk factors in autism. J Child Neurol, 14(6):388-394.
Connolly A M, Chez M G, Pestronk A et al. (1999) Serum autoantibodies to brain in Landau-Kleffner variant, autism, and other neurologic disorders. J Pediatr, 134(5):607-613.
Fatemi S H, Halt A R, Stary J M et al. (2002) Glutamic acid decarboxylase 65 and 67 kDa proteins are reduced in autistic parietal and cerebellar cortices. Biol Psychiatry, 52(8):805-810.
Folstein S and Rutter M (1977) Infantile autism: a genetic study of 21 twin pairs. J Child Psychol Psychiatry Allied Disciplines, 18:297-321.
Folstein SE and Rosen-Sheidley B R (2001) Genetics of autism: complex aetiology for a heterogeneous disorder. Nat Rev Genet, 2:943-955.
Gillberg C (1998) Chromosomal disorders and autism. J Autism Dev Disord, 28(5):415-425.
Gillberg C and Coleman M (2000) The biology of the autistic syndromes, 3^{rd} edn London: MacKeith Press.
Gillberg C and Wing L (1999) Autism: not an extremely rare disorder. Acta Psychiatr Scand, 99:339-406.
Hugot J P, Chamaillard M, Zouali H et al. (2001) Association of NOD2 leucine-rich repeat variants with susceptibility to Crohn's disease. Nature 411(6837):599-603.
Hussman JP (2001) Suppressed GABAergic inhibition as a common factor in suspected etiologies of autism. J Autism Dev Disord, 31(2):247-248.
Jamain S, Quach H, Betancur C et al. (2003) Mutations of the X-linked genes encoding neuroligins NLGN3 and NLGN4 are associated with autism. Nat Genet, 34(1):27-29.
Jorde L B, Hasstedt S J, Ritvo E R et al. (1991) Complex segregation analysis of autism. Am Hum Genet, 49(5):932-938.
Jorde L B, Mason-Brothers A, Waldmann R et al. (1990) The UCLA-University of Utah epidemiologic survey of autism: genealogical analysis of familial aggregation. Am J Med Genet, 36(1):85-88.
Kanner L (1943) Autistic disturbances of affective contact. Nervous Child, 2:217-250.
Lander E and Kniglyak L (1995) Genetic dissection of complex traits: guidelines for interpreting and reporting linkage results. Nat Genet, 11(3):241-247.
Le Couteur A, Rutter M, Lord C et al. (1989) Autism diagnostic interview: a standardized investigator-based instrument. J Autism Dev Disord, 19(3):363-387.
Lesage S, Zouali H, Cezard Jp et al. (2002) CARD15/NOD2 mutational analysis and genotype-phenotype correlation in 612 patients with inflammatory bowel disease. Am J Hum Genet. 70(4):845-857.
Lord C, Rutter M, Le Couteur A (1994) Autism Diagnostic Interview-Revised: a revised version of a diagnostic interview for caregivers of individuals with possible pervasive developmental disorders. J Autism Dev Disord, 24(5):659-685.
Nelson KB (1991) Prenatal and perinatal factors in the etiology of autism. Pediatrics, 87(5 Pt 2):761-766.
Nilsson M, Waters S, Waters N et al. (2001) A behavioural pattern analysis of hypoglutamatergic mice—effects of four different antipsychotic agents. J Neural Transm, 108(10):1181-1196.
Ogura Y, Bonen D K, Inohara N et al. (2001) A frameshift mutation in NOD2 associated with susceptibility to Crohn's disease. Nature 411(6837):603-606
Purcell A E, Jeon O H, Zimmerman A W et al. (2001) Postmortem brain abnormalities of the glutamate neurotransmitter system in autism. Neurology, 57(9):1618-1628.
Rioux J D, Daly M J, Silverberg M S et al. (2001) Genetic variation in the 5q31 cytokine gene cluster confers susceptibility to Crohn disease. Nat Genet. 29(2): 223-228.
Rioux J D, Silverberg M S, Daly M J (2000) Genomewide search in Canadian families with inflammatory bowel disease reveals two novel susceptibility loci. Am J Hum Genet. 66(6):1863-1870.
Rodier P and Hyman S (1998) Early environmental factors in autism. Mental Retard Dev Disord Res Rev, 4:121-128.
Singh V K, Warren R P, Odell J D et al. (1993) Antibodies to myelin basic protein in children with autistic behavior. Brain Behav Immun, 7(1):97-103.
Smalley S L (1997) Genetic influences in childhood-onset psychiatric disorders: autism and attention-deficit/hyperactivity disorder. Am J Hum Genet, 60(6):1276-1282.
Steffenburg S, Gillberg C, Hellgren L et al. (1989) A twin study of autism in Denmark, Finland, Iceland, Norway and Sweden. J Child Psychol Psychiatry, 30(3):405-416.
Szatmari P, Jones M B, Zwaigenbaum L et al. (1998) Genetics of autism: overview and new directions. J Autism Dev Disord, 28(5):351-368.
Weizman A, Weizman R, Szekely G A et al. (1982) Abnormal immune response to brain tissue antigen in the syndrome of autism. Am J Psychiatry, 139(11):1462-1465.

1-17. (canceled)

18. A method of detecting the presence of or predisposition to autism, or to an autism spectrum disorder, the method comprising (i) providing a sample from the subject and (ii) detecting the presence of an alteration in the SLC6A1 or SLC6A11 gene locus in the sample.

19. The method of claim 18, wherein the presence of an alteration in the SLC6A1 or SLC6A11 gene locus is detected by sequencing, selective hybridisation, or selective amplification.

20. The method of claim 18, wherein the alteration is one or several SNPs or a haplotype of SNPs associated with autism.

21. The method of claim 18 wherein, the alteration is in the SLC6A1 gene locus.

22. The method of claim 18, wherein the alteration is in the SLC6A11 gene locus.

23. The method of claim 20, wherein the alteration is in the SLC6A1 gene locus.

24. The method of claim 20, wherein the alteration is in the SLC6A11 gene locus.

25. A method of selecting biologically active compounds on autism, or autism spectrum disorders, said method comprising contacting a test compound with a SLC6A1 or SLC6A11 polypeptide or gene or a fragment thereof and determining the ability of the test compound to bind the SLC6A1 or SLC6A11 polypeptide or gene or a fragment thereof.

26. The method of claim 25, when the test compound is contacted with the SLC6A1 polypeptide or gene or a fragment thereof.

27. The method of claim 26, when the test compound is contacted with the SLC6A11 polypeptide or gene or a fragment thereof.

28. A method of selecting biologically active compounds on autism, or autism spectrum disorders, the method comprising contacting a recombinant host cell expressing a SLC6A1 or SLC6A11 polypeptide with a test compound, and determining the ability of the test compound to bind the SLC6A1 or SLC6A11 polypeptide and to modulate the activity of SLC6At or SLC6A11 polypeptide.

29. The method of claim 28, wherein the recombinant host cell expressing a SLC6A1 polypeptide is contacted with the test compound.

30. The method of claim 28, wherein the recombinant host cell expressing a SLC6A11 polypeptide is contact with the test compound.

31. A method of selecting biologically active compounds on autism, or autism spectrum disorders, the method comprising contacting a test compound with a SLC6A1 or SLC6A11 gene and determining the ability of the test compound to modulate the expression of the SLC6A1 or SLC6A11 gene.

32. The method of claim 31, wherein the test compound is contacted with the SCL6A1 gene.

33. The method of claim 31, wherein the test compound is contacted with the SCL6A11 gene.

34. A method of selecting biologically active compounds on autism, or autism spectrum disorders, said method comprising contacting a test compound with a recombinant host cell comprising a reporter construct, the reporter construct comprising a reporter gene is under the control of a SLC6A1 or SLC6A11 gene promoter, and selecting the test compounds that modulate expression of the reporter gene.

35. The method of claim 34, wherein the reporter gene is under the control of the SLC6A1 gene promoter.

36. The method of claim 34, wherein the reporter gene is under the control of the SLC6A11 gene promoter.
